(19) United States
(12) Patent Application Publication
(10) Pub. No.: US 2007/0043012 A1
(43) Pub. Date: Feb. 22, 2007

(54) Methods to enhance chemotherapy

(76) Inventor: Gary J. Bridger, Bellingham, WA (US)

Correspondence Address: MORRISON & FOERSTER LLP, 12531 HIGH BLUFF DRIVE, SUITE 100, SAN DIEGO, CA, 92130-2040, US

(21) Appl. No.: 11/507,717

(22) Filed: Aug. 21, 2006

Related U.S. Application Data
(60) Provisional application No. 60/709,978, filed on Aug. 19, 2005.
(60) Provisional application No. 60/734,736, filed on Nov. 8, 2005.

(51) Int. Cl. A61K 31/395

(52) U.S. Cl. 514/183

(57) ABSTRACT

Antagonists of the interaction of CXCR4 receptor with its ligand enhance the effectiveness of chemotherapeutic methods in subjects afflicted with myeloid or hematopoietic malignancies.

### RELATED APPLICATIONS

This application claims benefit of U.S. provisional applications Ser. Nos. 60/709,978, filed 19 Aug. 2005, and 60/734,736, filed 8 Nov. 2005. The contents of these documents are incorporated herein by reference in their entirety.

### TECHNICAL FIELD

The invention is in the field of treating hematopoietic-related cancers. More particularly, the invention concerns methods to enhance chemotherapy of such conditions.

### BACKGROUND ART

A common approach to hematopoietic-related cancers, such as myeloid leukemias and lymphoid leukemias, is a session of chemotherapy to destroy the malignant cells combined with transplantation of hematopoietic progenitor cells either of autogeneic or allogeneic origin. It is believed that the lack of success often experienced with this treatment regimen is due to failure of the chemotherapy to completely eliminate the malignant hematopoietic cells or their precursors. The present invention improves this method by coupling it with administration of a compound that enhances the effect of chemotherapy with respect to these residual malignant or pre-malignant cells.

The compounds useful in the method of the invention are antagonists of the CXCR4 receptor that prevent its interaction with the cytokine stromal cell derived factor-1 (SDF-1). Many such agents are known in the art. Such agents are disclosed, for example, in U.S. Pat. Nos. 5,021,409; 6,001,826; 5,583,131; 5,698,546; 5,817,807 and 6,506,770 incorporated herein by reference, and in PCT publications WO 92/16494; WO 93/12096; WO 95/18808; WO 00/02870; WO 00/56729; WO 01/44229; WO 02/22600; WO 02/22599; WO 02/34745, WO 03/055876; WO 04/091518 and WO 04/093217, also incorporated by reference.

We have previously found, and have disclosed in PCT publication WO 02/58653, that the certain CXCR4 antagonists, in particular, AMD3100, have the effect of increasing the white blood cell count. We have also found, and have disclosed in PCT publication WO 03/011277, that these antagonists have the effect of mobilizing progenitor cells and/or stem cells from the bone marrow to the circulating blood.

The chemokine receptor CXCR4 and its natural ligand SDF-1 appear to be important in the process of hematopoiesis (for reviews see Maekawa, T., et al., Internal Med. (2000) 39:90-100; Nagasawa, T., et al., Int. J. Hematol. (2000) 72:408-411). For example, CXCR4 or SDF-1 knock-out mice exhibit hematopoietic defects (Ma, Q., et al., Proc. Natl. Acad. Sci USA (1998) 95:9448-9453). It appears that SDF-1 is able to control the positioning and differentiation of cells bearing CXCR4 receptors whether these cells are stem cells (i.e., cells which are CD34+) or are progenitor cells (which result in formation of specified types of colonies in response to particular stimuli).

It appears that, within the microenvironment of the bone marrow, SDF-1 acts as a potent chemoattractant for immature and mature hematopoietic cells, and thus expression of CXCR4 on leukemic progenitor cells and leukemia cells may contribute to homing them to the bone marrow microenvironment. Elevated CXCR4 levels are detected on leukemic cells from patients with B chronic lymphocytic leukemia (B-CLL) (Mohle, R., et al., Leukemia (1999) 13:1954-1959). It further appears that autocrine secretion of SDF-1 by blood-derived adherent nurse-like cells in chronic lymphocytic leukemia (CLL) protects leukemic B cells from spontaneous apoptosis (Burger, J. A., et al., Blood (2000) 96:2655-2663). Enhanced levels were not detected on leukemic cells from patients with T-ALL or leukemic cells from patients with AML according to Mohle, et al., supra; Voermans, C., et al., Leukemia (2002) 16:650-657; Bradstock, K. F., et al., Leukemia (2000) 14:882-888; Dialynas, D. P., et al., Stem Cells (2001) 19:443-452; Shen, W., et al., Exp. Hematol. (2001) 29:1439-1447. However, it appears that expression levels of CXCR4 vary among various types of AML as reported by Rombouts, E. J., et al., Blood (2004) 104:550-557; Fukuda, S., et al., Blood (2005) 105:3117-3126. CXCR4 is also reported to mediate homing and engraftment of pre-B-ALL and AML cells to bone marrow, although other factors may be involved (Shen, et al., supra; Tavor, S., et al., Cancer Res. (2004) 64:2817-2824.). It was recently shown, in an in vitro context, that AMD3100 blocked SDF-1 induced chemotaxis of pre-B-ALL cells into bone marrow stroma layers, and enhanced the cytotoxic and antiproliferative effects of vincristine and dexamethasone (Juarez, J., et al., Leukemia (2003) 17:1294-1300.) These studies suggest that SDF-1/CXCR4 interactions are involved in the microenvironmental regulation of leukemic cells and such interaction plays a role in the resistance of residual, post-chemotherapy AML exposure to additional chemotherapeutic agents.

There is a need to mobilize pre-cancerous or cancerous cells from the bone marrow and into the peripheral blood system, where these cells can be exposed to chemotherapeutic agents. The current invention addresses such need by use of inhibitors of the CXCR4 receptor to potentiate the effects of standard chemotherapeutic agents, through the release and/or rapid movement of pre-leukemic cells and leukemic cells from the microenvironment of the bone marrow and into circulating blood prior to, or during, or after treatment by chemotherapy.

This invention may be used to treat subjects that may or may not require transplantation.

Citation of the above documents is not intended as an admission that any of the foregoing is pertinent prior art. All statements as to the date or representation as to the contents of these documents is based on the information available to the applicants and does not constitute any admission as to the correctness of the dates or contents of these documents, and not intended to be bound by any theory or hypothesis described in these documents. Further, all documents referred to throughout this application are incorporated in their entirety by reference herein.

### DISCLOSURE OF THE INVENTION

The invention is directed to methods of treating animal subjects, in particular, veterinary and human subjects, with chemotherapeutic methods while also administering a CXCR4 antagonist that enhances the effect of the chemotherapy.

Thus, in one aspect, the invention is directed to a method to treat a subject afflicted with a hematopoietic malignancy, such as a lymphoma, a myeloma, or a leukemia, which method comprises administering one or more CXCR4 antagonists and one or more chemotherapeutic agents. The CXCR4 antagonist(s) may be administered before, during, and/or after chemotherapeutic regimens are administered.

In another aspect, the invention is directed to pharmaceutical or veterinary compositions comprising a CXCR4 antagonist for use in the method of the invention. These compositions comprise one or more CXCR4 antagonists along with suitable pharmaceutically or veterinary acceptable excipients.

Some of the antagonists useful in the invention are those disclosed in U.S. Pat. Nos. 5,021,409; 6,001,826; 5,583,131; 5,698,546; 5,817,807 and 6,506,770 incorporated herein by reference, and in PCT publications WO 92/16494; WO 93/12096; WO 95/18808; WO 00/02870; WO 00/56729; WO 01/44229; WO 02/22600; WO 02/22599; WO 02/34745, WO 03/055876; WO 04/091518 and WO 04/093817, also incorporated by reference. Peptide-based antagonists are described in WO 2001/85196; WO 2000/09152 and WO 99/47158. The use of antibodies as inhibitors of CXCR4 interacting with its ligand are disclosed in WO 99/50461. Other compounds include T22 (Murakami, T., et al., J. Exp. Med., 186:1389-1393 (1997)), ALX40-4C (Doranz, B. J., et al., J. Exp. Med., 186, 1395-1400 (1997)); Donzella, G. A., Nat. Med., 4, 72-77 (1998)), and the like. As to the methods for preparation of these substances, they can, for example, be found in J. Exp. Med., 186, 1189-1191 (1997) with any conventional modifications.

### MODES OF CARRYING OUT THE INVENTION

The invention employs compounds that inhibit the binding of SDF-1 to CXCR4 (CXCR4 antagonists). While not wishing to be bound by any theory, the compounds which inhibit the binding of SDF-1 to CXCR4 effect enhancement of chemotherapy by virtue of such inhibition, by depriving the malignant or pre-malignant cells from the protection of the stromal cells of the bone marrow.

As used herein, the term “pre-malignant cells” refers to cells that can form malignant hematopoietic or myeloid cells. The malignant hematopoietic or myeloid cells are those which characterize the conditions of myeloma, leukemia, and lymphoma. Particular forms of these diseases include acute myelitic leukemia (AML), acute lymphatic leukemia (ALL), multiple myeloma (MM), chronic myelogenous leukemia (CML), hairy cell leukemia (HCL), acute promyelocytic leukemia (APL), Chronic lymphocytic leukemia (CLL) and various lymphomas.

Chemotherapeutic compounds, or agents which may be used in the methods whose effectiveness is enhanced by the methods of the invention include carboplatin, carmustine, chlorambucil, dacarbazine, ifosfamide, lomustine, mechlorethamine, procarbazine, pentostatin, (2′deoxycoformycin), etoposide, teniposide, topotecan, vinblastine, vincristine, paclitaxel, dexamethasone, methylprednisolone, prednisone, all-trans retinoic acid, arsenic trioxide, interferon-alpha, rituximab (Rituxan®), gemtuzumab ozogamicin, imatinib mesylate, cytarabine (cytosine arabinoside, Ara-C, Cytosar-U), melphalan, busulfan (Myleran®), thiotepa, bleomycin, platinum (cisplatin), cyclophosphamide, Cytoxan®, daunorubicin, doxorubicin, idarubicin, mitoxantrone, 5-azacytidine, cladribine, fludarabine, hydroxyurea, 6-mercaptopurine, methotrexate, 6-thioguanine, and many others.

A wide variety of chemotherapeutic methods are available in the art. The invention herein employs these standard methods or variations thereof but, in addition, provides for administration of the CXCR4 antagonists to enhance the effect of such methods. Preferably, these antagonists are administered prior to and/or concomitant with subjecting the patient to such methods. Administration may continue after the method has ceased as well, if desired. Dosage levels and mode of administration are interdependent. When given subcutaneously, for example, the dosage levels are in the range of 50 μg/kg-1 mg/kg, preferably 200 μg/kg-500 μg/kg. Dosage levels using oral administration may be higher and intravenous administration somewhat lower.

In some embodiments, the CXCR4 antagonist is of the formula
Z-linker-Z′ (1)

wherein Z is a cyclic polyamine containing 9-32 ring members of which 2-8 are nitrogen atoms, said nitrogen atoms separated from each other by at least 2 carbon atoms, and wherein said heterocycle may optionally contain additional heteroatoms besides nitrogen and/or may be fused to an additional ring system;

or Z is of the formula

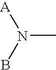

wherein A comprises a monocyclic or bicyclic fused ring system containing at least one N and B is H or an organic moiety of 1-20 atoms;

Z′ may be embodied in a form as defined by Z above, or alternatively may be of the formula
—N(R)—(CR₂)ₙ—X

wherein each R is independently H or straight, branched or cyclic alkyl (1-6C),

n is 1 or 2, and

X is an aromatic ring, including heteroaromatic rings, or is a mercaptan;

or Z′ may be absent and the compound of formula 1 terminates with the moiety defined below as a linker;

“linker” represents a bond, alkylene (1-6C) or may comprise aryl, fused aryl, oxygen atoms contained in an alkylene chain, or may contain keto groups or nitrogen or sulfur atoms.

Specific forms of the compounds of formula (1) are discussed below.

In compounds of formula (1), some embodiments of Z and Z′ are cyclic polyamine moieties having from 9-24C that include 3-5 nitrogen atoms, for example, 1,5,9,13-tetraazacyclohexadecane; 1,5,8,11,14-pentaazacyclohexadecane; 1,4,8,11-tetraazacylotetradecane; 1,5,9-triazacyclododecane; 1,4,7,10-tetraazacyclododecane; and the like, including such cyclic polyamines which are fused to an additional aromatic or heteroaromatic rings and/or containing a heteroatom other than nitrogen incorporated in the ring. Embodiments wherein the cyclic polyamine contains a fused additional cyclic system or one or more additional heteroatoms are described in U.S. Pat. No. 5,698,546 and WO 01/44229 incorporated hereinabove by reference. Other embodiments are 3,7,11,17-tetraazabicyclo(13.3.1)heptadeca-1(17),13,15-triene; 4,7,10,17-tetraazabicyclo(13.3.1)heptadeca-1(17),13,15-triene; 1,4,7,10-tetraazacyclotetradecane; 1,4,7-triazacyclotetradecane; and 4,7,10-triazabicyclo(13.3.1)heptadeca-1(17),13,15-triene.

Some embodiments of the compound of the formula (1) include 2,2′-bicyclam; 6,6′-bicyclam; the embodiments set forth in U.S. Pat. Nos. 5,021,409, and 6,001,826, and in particular 1,1′-[1,4-phenylene-bis(methylene)]-bis-1,4,8,11-tetraazacyclotetradecane, set forth in U.S. Pat. No. 5,583,131, and designated herein AMD3100.

In some embodiments, Z is 1,4,8,11-tetraazacyclotetradecane, the linker is 1,3- or 1,4-phenylene-bis(alkylene) in particular 1,4-phenylene-bis(methylene) and Z′ is —NR(CR₂)ₙ—X, where X is pyridine, and in particular wherein Z′ is NHCH₂CH₂-pyridine. In some embodiments, the compound is AMD3465 which is N-[1,4,8,11-tetraazacyclotetradecanyl-(1,4-phenylene-bis-(methylene)]-2-aminoalkylpyridine or substituted forms thereof.

When Z′ is other than a cyclic polyamine as defined in Z, some embodiments are set forth in U.S. Pat. Nos. 5,817,807 and 6,506,770 also incorporated herein by reference.

Some forms where

Z is of the formula

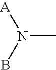

wherein A comprises a monocyclic or bicyclic fused ring system containing at least one N and B is H or an organic moiety of 1-20 atoms are disclosed in WO 00/56729; WO 02/22600; WO 02/34745; WO 02/22599 and WO 03/55876 cited above and all incorporated herein by reference.

In one embodiment, as set forth in WO 03/55876, A is 5,6,7,8-tetrahydroquinoline-8-yl and B is 1H-benzimidazol-2-yl methyl. In some of these embodiments, Z′ is absent and the linker is an omega aminoalkyl group. Thus, one illustrative compound is AMD11070 which is N¹-(1H-benzimidazol-2-yl methyl)-N¹-(5,6,7,8-tetrahydroquinolin-8-yl)butane-1,4-diamine. Thus, important embodiments include AMD11070 and substituted forms thereof.

Forms of the linker moiety include those wherein the linker is a bond, or wherein the linker includes an aromatic moiety flanked by alkylene, preferably methylene moieties. Linking groups include the methylene bracketed forms of 1,3-phenylene, 2,6-pyridine, 3,5-pyridine, 2,5-thiophene, 4,4′-(2,2′-bipyrimidine); 2,9-(1,10-phenanthroline) and the like. A particularly preferred linker is 1,4-phenylene-bis-(methylene).

Additional compounds that are CXCR4 antagonists are disclosed in U.S. applications Ser. No. 10/823,494 filed 12 Apr. 2004 and 10/831,098 filed 22 Apr. 2004 and 11/012,002 filed 13 Dec. 2004, incorporated herein by reference.

CXCR4 inhibitors that may be used to practice the methods of the invention also include but are not limited to CTCE-0214; CTCE-9908; CP-1221 (linear peptides, cyclic peptides, natural amino-acids, unnatural amino acids, and peptidomimetic compounds); T140 and analogs; 4F-benzoyl-TN24003; KRH-1120; KRH-1636; KRH-2731; polyphemusin analogue; ALX40-4C; or those described in WO 01/85196; WO 99/50461; WO 01/94420; WO 03/090512, each of which is incorporated by reference herein.

Methods to synthesize the compounds useful in the method of the invention are set forth in the U.S. patents and applications above as well as U.S. Pat. No. 6,489,472 and U.S. application Ser. No. 11/077,896 filed 11 Mar. 2005, incorporated herein by reference. Additional CXCR4 inhibitors are set forth in Appendix A.

As noted above, AMD3100 is an exemplary antagonist with the CXCR4 chemokine receptor (Gerlach, et al., J. Biol. Chem. (2001) 276:14153-14160). This compound interferes with the binding of bone marrow stromal cell derived SDF-1 with CXCR4 on stem cells which leads to the release of hematopoietic stem cells from bone marrow into the circulation (Broxmeyer, et al., Blood (2001) 98:811a (Abstract)).

The compounds of the invention may be prepared in the form of prodrugs, i.e., protected forms which release the compounds of the invention after administration to the subject. Typically, the protecting groups are hydrolyzed in body fluids such as in the bloodstream thus releasing the active compound or are oxidized or reduced in vivo to release the active compound. A discussion of prodrugs is found in Smith and Williams Introduction to the Principles of Drug Design, Smith, H. J.; Wright, 2^{nd} ed., London (1988).

Compounds useful in the invention which are amines, may be administered or prepared in the forms of their acid addition salts or metal complexes thereof. Suitable acid addition salts include salts of inorganic acids that are biocompatible, including HCl, HBr, sulfuric, phosphoric and the like, as well as organic acids such as acetic, propionic, butyric and the like, as well as acids containing more than one carboxyl group, such as oxalic, glutaric, adipic and the like. Typically, at physiological pH, the compounds of the invention will be in the forms of the acid addition salts.

Compounds useful in the invention that are carboxylic acids or otherwise acidic may be administered or prepared in forms of salts formed from inorganic or organic bases that are physiologically compatible. Thus, these compounds may be prepared in the forms of their sodium, potassium, calcium, or magnesium salts as appropriate or may be salts with organic bases such as caffeine or ethylamine. These compounds also may be in the form of metal complexes.

When prepared as purified forms, the compounds may also be crystallized as the hydrates or other solvates. Those forms of the compounds used in the invention that contain chiral centers may be optically pure or may contain a mixture of stereoisomers, including racemic mixtures or mixtures of varying optical purity.

The CXCR4 antagonists may be formulated for administration to animal subject using commonly understood formulation techniques well known in the art. Formulations which are suitable for particular modes of administration and for compounds useful in the invention may be found in Remington's Pharmaceutical Sciences, latest edition, Mack Publishing Company, Easton, Pa.

Preferably, the CXCR4 antagonists are administered by injection, most preferably by intravenous injection, but also by subcutaneous or intraperitoneal injection, and the like. Additional parenteral routes of administration include intramuscular and intraarticular injection. For intravenous or parenteral administration, the compounds are formulated in suitable liquid form with excipients as required. The compositions may contain liposomes or other suitable carriers. For injection intravenously, the solution is made isotonic using standard preparations such as Hank's solution.

Besides injection, other routes of administration may also be used. The compounds may be formulated into tablets, capsules, syrups, powders, or other suitable forms for administration orally. By using suitable excipients, these compounds may also be administered through the mucosa using suppositories or intranasal sprays. Transdermal administration can also be effected by using suitable penetrants and controlling the rate of release.

The formulation and route of administration chosen will be tailored to the individual subject, the nature of the condition to be treated in the subject, and generally, the judgment of the attending practitioner.

Suitable dosage ranges for the CXCR4 antagonists vary according to these considerations, but in general, the compounds are administered in the range of about 0.1 μg/kg-5 mg/kg of body weight; preferably the range is about 1 μg/kg-500 μg/kg up to 1 mg/kg of body weight. For a typical 70-kg human subject, thus, the dosage range is from about 0.7 μg-350 mg. Dosages may be higher when the compounds are administered orally or transdermally as compared to, for example, i.v. administration.

The CXCR4 antagonists may be administered as a single bolus dose, a dose over time, as in i.v. or transdermal administration, or in multiple dosages. The CXCR4 antagonists may be administered along with other factors that aid in mobilization, or other factors that are nutritional or therapeutically beneficial. The additional factor(s) may be administered in the same composition, in different compositions but simultaneously, or in a tandem protocol with the administration of the CXCR4 antagonist. Among additional factors that can be included are recombinant G-CSF (Neupogen®, Granocyte®/Neutrogin®, and Stemgen®), a covalent conjugate of recombinant G-CSF (Neulasta®), granulocyte-macrophage colony stimulating factor (GM-CSF) (such as Leukine®, and Leucomax®), Interleukin-1 (IL-1), Interleukin-3 (IL-3), Interleukin-8 (IL-8), PIXY-321 (GM-CSF/IL-3 fusion protein), macrophage inflammatory protein, stem cell factor, and thrombopoietin, as well as antibiotics, vitamins, herbal extracts, anti-inflammatories, nutrients, antipyretics, analgesics, cyclophosphamide and the like.

As noted, the compounds as administered in conjunction with chemotherapeutic methods. These methods are those generally employed in the treatment of the hematopoietic or myelitic malignancies that are subject to treatment by the method of the invention. A wide variety of such methods is known in the art.

Subjects that will respond favorably to the method of the invention include medical and veterinary subjects generally, including human patients. Among other subjects for whom the methods of the invention is useful are cats, dogs, large animals, avians such as chickens, and the like, other than standard research rodents such as laboratory mice, rabbits, or rats. In general, any subject that exhibits a hematopoietic or myelitic malignancy would benefit from the methods of the invention.

A wide variety of chemotherapeutic protocols is employed, many of such protocols involving combinations of drugs administered simultaneously or in tandem. The CXCR4 antagonists may be administered at various points in the simultaneous or tandem protocols. For example, one protocol for AML involves combinations of busulfan and fludarabine. These drugs are administered intravenously. The CXCR4 antagonist may be administered several hours before the first administration of this drug which is repeated over several days. The CXCR4 antagonist may be administered each day prior to or during the administration of the fludarabine, or only typical, busulfan is administered subsequent to fludarabine over several days, and the CXCR4 antagonist may be administered each day along with, before, or after the administration of the busulfan, one administration before, during or after treatment may be required.

Various combinations of the foregoing agents are used in such protocols, and the timing and frequency of CXCR4 administration is subject to routine optimization, well within ordinary skill.

Having now generally described the invention, the same will be more readily understood through reference to the following examples which are provided by way of illustration, and are not intended to be limiting of the present invention, unless specified.

### EXAMPLE 1

This example describes a murine model of human acute promyelocytic leukemia (APL) used to determine the effect of a CXCR4 antagonist on the mobilization of APL cells into the peripheral blood, and on their sensitivity to chemotherapeutic agents known to affect the proliferation of these cells. Murine APL cells were generated by “knocking-in” the PML-RARα cDNA from human APL into the murine cathepsin G locus (Westervelt, et al., PubMed (2003) 102(5):1857-1865) that resulted in the overexpression in the murine promyelocyte compartment. Mouse APL cells, after injection into syngeneic recipients, home preferentially to the bone marrow microenvironment in a manner similar to what is observed in human AML, and expands there for 20-30 days after circulating in high numbers in the peripheral blood. This ultimately leads to death of the animals by 50 to 80 plus days.

Using a murine model of APL, one may determine whether leukemic cells are “mobilized” in a similar manner to normal stem cells after treatment with a test compound, such as AMD3100, AMD3465, AMD11070, and other compounds described herein. In one instance, AMD3100 (5 mg/kg) injected immediately at the same time when APL cells were injected did not have any impact on the engraftment (short or long term) of either normal bone marrow stem cells or the leukemic cells. However, where AMD3100 was administered 11 days after APL injection, a rapid mobilization of the leukemic cells was observed. Forty percent (⅖) of mice that received a single dose of AMD3100 on day+11 after APL injection died 2 to 4 hours after the administration of AMD3100. It was observed that administration of AMD3100 on day+11 induced a 3-fold increase in total white blood cell (WBC) counts, and a 10-fold increase in the leukemic blasts into peripheral blood.

When AMD3100 was administered concomitantly with cytarabine (200 mg/kg) on day+11 into mice, this treatment significantly prolonged the overall survival of mice, compared with mice treated only with cytarabine. Based on the observed results, it may be possible that tumor resistance may be overcome by potentiating the effects of chemotherapeutic agents by mobilizing tumor cells from the bone marrow into the peripheral blood.

### EXAMPLE 2

### Clinical Study

The in vivo effect of the CXCR4 antagonist AMD3100 was studied in three patients with AML, who had insufficient mobilization of CD34+ cells for autologous stem cell transplantation with G-CSF and/or Cytoxan®. The combination of G-CSF and AMD3100 (for 3-4 days) resulted in massive mobilization of leukemic cells into the circulation in a time-dependent fashion, as determined by flow cytometry and interphase FISH analysis of their respective cytogenetic abnormalities.

| | Cytogenetics, | % (+) cells | % (+) cells | Apheresis |
|---|---|---|---|---|
| Patient# | FCM | Day 2 | Day 4/5 | CD34 × 10⁶/kg |
| 1 | Trisomy 21 | | 22.6 | 57.0 |
| | FCM CD7/33 | | 22.0 | |
| 2 | Trisomy 9 | 28.6 | 68.6 | 4.8 |
| | Inv 16 | 29.0 | 75.8 | |
| | FCM CD13/33 | | 74.0 | |
| 3 | Mono 17 | 40.4 | 53.4 | 8.7 |
| | 5q31 | 37.5 | 49.6 | |
| | FCM CD13/33 | | 50.0 | |

### EXAMPLE 3

### In Vitro Data

In a previously demonstrated study, it was shown that stroma/leukemia interactions mediate protection of leukemic cells from chemotherapy-induced apoptosis (Konopleva, M., Leukemia (2002):1713-1724). Co-culture systems of AML cells with stromal cells in vitro showed stromal cells significantly protected leukemic cells (p<0.01). Application of AMD3465 decreased stroma-mediated protection from AraC and busulfan apoptosis and downregulated AKT signaling in AML cells.

### EXAMPLE 4

### Animal Model

In a murine model of luciferase labeled Baf-FLT3ITD leukemias, AMD3465 induced massive dissemination of leukemia, which was abrogated by treatment with sorafenib, a potent FLT3ITD inhibitor (Zhang, ASH 2006).

### Appendix A

Exemplary CXCR4 antagonists of Formula 1 include compounds of formula (1A):
V—CR₂—Ar¹—CR₂NR—(CR₂)ₓ—Ar² (1A)

wherein V is a substituted heterocycle of 9-24 members containing 2-4 optionally substituted amine nitrogen atoms spaced from each other by 2 or more optionally substituted carbon atoms, and which heterocycle may optionally comprise a fused aromatic or heteroaromatic ring, and wherein

(a) said heterocycle contains at least one O or S, said O or S spaced from any adjacent heteroatom by at least 2 carbon atoms, and wherein said S is optionally oxidized or

(b) at least one carbon atom in said ring is substituted by an electron-withdrawing substituent, or

(c) both (a) and (b);

and wherein each R is independently H or a straight chain, branched or cyclic alkyl containing 1-6C;

x is 0-4;

Ar¹ is an unsubstituted or substituted aromatic or heteroaromatic moiety; and

Ar² is an unsubstituted or substituted aromatic or heterocyclic group.

In another embodiment of Formula 1, the CXCR4 antagonist has formula
V—CH₂—Ar¹—CH₂NR—CH₂—Ar²

wherein V is a heterocycle as defined in formula (1A), and wherein:

(a) said heterocycle is substituted with halo or ═O; or

(b) said heterocycle contains O or S; or

(c) both (a) and (b),

and wherein Ar¹ is unsubstituted 1,3 or 1,4-phenylene, R is H, methyl or ethyl and Ar² is unsubstituted phenyl or pyridinyl. Preferred embodiments of x are 0-2 and 1-2.

The heterocycle V may contain 3 N and at least one carbon atom in the heterocycle that is substituted by at least one fluoro substituent. The R moiety may independently be hydrogen or methyl. The number of (CR₂)ₓ groups may be 0-4, 0-2, or 1-2. The Ar¹ moiety may be 1, 3 or 1,4-phenylene. The Ar² moiety may be phenyl or pyridyl. The heterocycle V may be a 12-16 membered heterocycle, or may contain O or S as a ring member. The heterocycle V may also contain an oxidized sulfur as a ring member. In one example, at least one carbon in the heterocycle V is substituted by ═O.

Compounds of formula (1A), and methods of synthesizing such compounds are described in WO 01/44229, incorporated herein by reference.

Related to these compounds having formula (1B):
V—CR¹R²—Ar—CR³R⁴—N(R⁵)—(CR⁶R⁷)ₓ—R⁸ (1B)

wherein V is an optionally substituted 1,4,8,11-tetraazacyclotetra-decanyl, 4,7,10,17-tetraazabicyclo[13.3.1]heptadeca-1(17),13,15-trienyl, 1,4,7-triazacyclotetra-decanyl, 4,7,10-triazabicyclo[13.3.1]heptadeca-1(17),13,15-trienyl, 1,7-diazacyclotetradecanyl, or 4,10-diazabicyclo[13.31.1]heptadeca-1(17),13,15-trienyl system;

R¹ to R⁷ may be the same or different and are independently selected from hydrogen or straight, branched or cyclic C₁₋₆ alkyl;

R⁸ is pyridyl, pyrimidinyl, pyrazinyl, imidazolyl, thiophene-yl, thiophenyl, aminobenzyl, piperidinyl, purine, piperazinyl, phenylpiperazinyl, or mercaptan;

Ar is a phenylene ring optionally substituted at single or multiple positions with alkyl, aryl, amino, alkoxy, hydroxy, halogen, carboxyl and/or carboxamido; and

x is 1 or 2.

In the above formula (1B), the V moiety may be optionally substituted by hydroxyl, alkoxy, thiol, thioalkyl, halogen, nitro, carboxy, amido, sulfonic acid, and/or phosphate.

Compounds of Formula (1B), its pharmaceutically acceptable salts or metal complexes thereof, and methods of synthesizing such compounds are described in WO 00/02870, which is incorporated herein by reference.

Other CXCR4 inhibitors are of formula (1C):
V²—CR₉R₁₀—Ar² (1C)

wherein V² is an optionally substituted 1,4,8,11-tetraazacyclotetra-decanyl or 4,7,10,17-tetraazabicyclo [13.3.1]heptadeca-1(17),13,15-trienyl system;

R₉ and R₁₀ may be the same or different and are independently selected from hydrogen or straight, branched or cyclic C₁₋₆ alkyl;

Ar₂ is an aromatic or heterocyclic ring each optionally substituted at single or multiple positions with electron-donating or withdrawing groups and/or aromatic and heterocyclic groups and their alkyl derivatives thereof, and the acid addition salts and metal complexes.

In the above Formula (1C), Ar₂ may be optionally substituted with alkyl, aryl, amino, alkoxy, hydroxy, halogen, carboxyl and/or carboxamido. In particular examples, Ar₂ is optionally substituted with alkoxy, alkyl, or halogen.

Compounds having formula (1C), and methods of synthesizing the same, are described in WO 00/02870, incorporated herein by reference.

Other CXCR4 antagonists are of formula (1D):
V—R-A-R′—W (1D)

wherein V and W are independently cyclic polyamine moieties having from 9 to 32 ring members and from 3 to 8 amine nitrogens in the ring spaced by 2 or more carbon atoms from each other, and having one or more aromatic or heteroaromatic rings fused thereto,

A is an aromatic or heteroaromatic moiety when V and W have one or more aromatic or heteroaromatic moieties fused thereto, with or without an additional heteroatom other than nitrogen incorporated in the ring, or A is an aromatic or heteroaromatic moiety when V and W contain a heteroatom other than nitrogen incorporated in the ring without having one or more aromatic or heteroaromatic moieties fused thereto,

and R and R′ are each a substituted or unsubstituted alkylene chain or heteroatom-containing chain which spaces the cyclic polyamines and the moiety A.

In the above Formula (1D), R and R′ may each be methylene. In one example, A is 1,3- or 1,4-phenylene. In another example, each V and W is an unsubstituted or substituted tricyclic or bicyclic ring system containing only carbon and nitrogen atoms in the rings. One of the cyclic ring systems may be a 10 to 20 membered polyamine ring system having from 3 to 6 amine nitrogen atoms, and the ring system or systems is a fused benzyl or pyridinyl ring system.

Compounds having formula (1D), and methods of synthesizing such compounds, are described in U.S. Pat. No. 5,698,546, incorporated herein by reference.

Other CXCR4 antagonists are of formula (1E):
Z-R-A-R′—Y (1E)

where Z and Y are identical cyclic polyamine moieties having from 10 to 15 ring members and from 3 to 6 amine nitrogens in the ring spaced by 2 or more carbon atoms from each other, said amine nitrogens being the only ring heteroatoms,

A is an aromatic or heteroaromatic moiety other than quinoline,

R and R′ are each methylene linked to nitrogen atoms in Z and Y, the amine nitrogen atoms being otherwise unsubstituted.

In the above formula (1E), each moiety Z and Y may have 14 ring members and 4 amine nitrogens in the ring. Compounds having formula (1E), and methods of synthesizing such compounds, are described in U.S. Pat. No. 5,583,131, incorporated herein by reference.

The CXCR4 antagonist may be of formula (1F):
Z-(A)ₙ-Y (1F)

where Z and Y are independently cyclic polyamine moieties having from 9 to 32 ring members and from 3 to 8 amine nitrogen atoms in the ring,

A is a linking atom or group, and n is O or an integer from 1 to 6.

In the above formula (1F) each Z and Y moiety may have 10 to 24 ring members, or 12 to 18 ring members. Each Z and Y moiety may also have 4 to 6 amine nitrogen atoms in the ring. In one example, n is 0. In another example, A is methylene.

Compounds having formula (1F), and methods of synthesizing such compounds, are described in U.S. Pat. No. 5,021,409, incorporated herein by reference.

Other CXCR4 antagonists are of formula (2A):

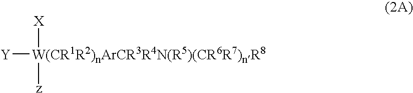

W is a nitrogen atom and Y is void, or W is a carbon atom and Y═H;

R¹ to R⁷ may be the same or different and are independently hydrogen or straight, branched or cyclic C₁₋₆ alkyl;

R⁸ is an optionally substituted heterocyclic group or an optionally substituted aromatic group

Ar is an aromatic or heteroaromatic ring optionally substituted at single or multiple, non-linking positions with electron-donating or withdrawing groups;

n and n′ are independently, 0-2;

X is a group of the formula:

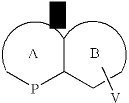

wherein, Ring A is an optionally substituted, saturated or unsaturated 5 or 6-membered ring, and P is an optionally substituted nitrogen atom and wherein any heteroatom in addition to P in ring A is N;

wherein Ring B is an optionally substituted 5 to 7-membered ring;

wherein Ring A or Ring B is bound to group W from any position through group V;

wherein V is a chemical bond or V is a (CH₂)_{n″} group (where n″=1-2), or V is a C═O group; and

wherein Z is selected from the group consisting of: a hydrogen atom; an optionally substituted C₁₋₆ alkyl group; an optionally substituted aromatic or heterocyclic group; an optionally substituted amino group; an optionally substituted C₁₋₆ alkylamino or C₃₋₇ cycloalkylamino group; and a substituted carbonyl group; or

the pharmaceutically acceptable acid addition salts thereof;

wherein said compound may be in any stereoisomeric form or present as a mixture of stereoisomeric forms thereof;

wherein Ring B is selected from the group consisting of: benzene and a 5 to 7-membered cycloalkyl ring; and the optionally substituted forms thereof.

The CXCR4 antagonists also include compounds of formula (2B):

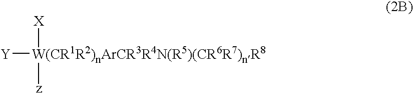

wherein, W is a nitrogen atom and Y is void;

R¹ to R⁷ may be the same or different and are independently hydrogen or straight, branched or cyclic C₁₋₆ alkyl;

R⁸ is an optionally substituted heterocyclic group or an optionally substituted aromatic group

Ar is an aromatic or heteroaromatic ring optionally substituted at single or multiple, non-linking positions with electron-donating or withdrawing groups;

n and n′ are independently, 0-2;

X is a group of the formula:

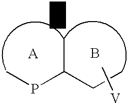

wherein, Ring A is an optionally substituted, saturated or unsaturated 5 or 6-membered ring, and P is an optionally substituted nitrogen atom and wherein any heteroatom in ring A or B is N;

wherein Ring B is an optionally substituted 5 to 7-membered ring;

wherein Ring A or Ring B is bound to group W from any position through group V;

wherein V is a chemical bond or V is a (CH₂)_{n″} group (where n″=1-2), or V is a C═O group; and

wherein Z is selected from the group consisting of: a hydrogen atom; an optionally substituted C₁₋₆ alkyl group; an optionally substituted aromatic or heterocyclic group; an optionally substituted amino group; an optionally substituted C₁₋₆ alkylamino or C₃₋₇ cycloalkylamino group; and a substituted carbonyl group; or the pharmaceutically acceptable acid addition salts thereof;

wherein said compound may be in any stereoisomeric form or present as a mixture of stereoisomeric forms thereof.

Compounds having formula (2A) and (2B) and methods for synthesizing such compounds are set forth in WO 00/56729, incorporated herein by reference.

Other CXCR4 antagonists are compounds of formula (3):

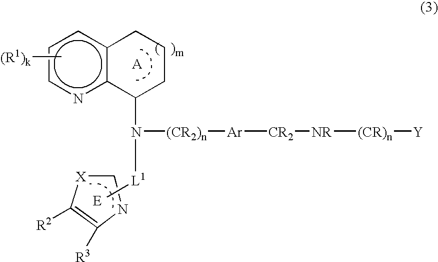

or the salts, prodrugs and stereochemical forms thereof, wherein:

Ring A optionally comprises a heteroatom selected from N, O and S;

the dotted lines represent optional unsaturation;

R¹ is halo, nitro, cyano, optionally substituted hydroxy, optionally substituted thiol, optionally substituted amino, carboxylate, carboxamide, sulfonate, sulfonamide, C2-4 alkanoyl, alkylsulfonyl, or aroyl;

R² and R³ are independently H, an optionally halogenated C1-4 alkyl, an optionally substituted aryl or heterocyclic group, or R² and R³ together with ring E may form a substituted or unsubstituted 5-7 membered ring;

k is 0-4;

m is 0-2;

L¹ is a covalent bond of C1-6 alkyl optionally containing N or O;

X is unsubstituted or substituted C, N; or O or S;

Ar is phenylene;

each n is independently 0-2;

each R is independently H or alkyl (1-6C); and

Y is a fused or unfused aromatic or heteroaromatic ring, or a 5-6 membered heterocyclic group.

The CXCR4 antagonists may also have formula (3A):

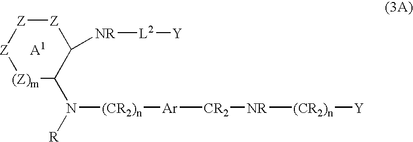

or the salts, prodrugs and stereochemical forms thereof, wherein:

R, m, n, Ar, and each Y are defined as in formula (3);

L² is a covalent bond or C1-6 alkyl optionally containing N or O;

and each Z is independently CR₂, NR, O or S, with the proviso that only two Z can be other than CR₂.

In the above formula (3A), L² may be methylene or ethylene. In one example, m is 1 and all Z embodiments are CR₂, particularly CH₂.

In the above formula (3A), each Y may be pyrimidyl, pyridyl, phenyl, benzimidazole or benzoxazole.

Other CXCR4 antagonists have formula (3B):

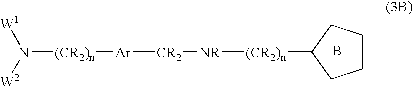

or the salts, prodrugs and stereochemical forms thereof, wherein:

W¹ is a monocyclic (5-6 membered) or fused bicyclic (8-12 membered) unsubstituted or substituted ring system containing at least one heteroatom selected from N, O and S;

W² is H, or is selected from the group consisting of: an optionally substituted C₁₋₆ alkyl group; a C₀₋₆ alkyl group substituted with an optionally substituted aromatic or heterocyclic group; an optionally substituted C₀₋₆ alkylamino or C₃₋₇ cycloalkylamino group; and an optionally substituted carbonyl group or sulfonyl;

Ar, R and n are defined as in Formula (3), and

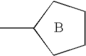

is a saturated or unsaturated 5-membered ring containing 1-2 heteroatoms selected from N, O and S.

Other CXCR4 antagonists have formula (3C):

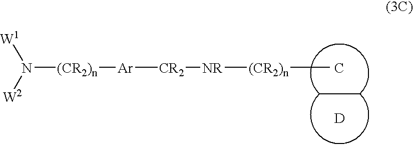

or the salts, prodrugs or stereochemical forms thereof, wherein:

W¹ is phenyl, pyridyl, pyridimyl, imidazolyl, thiophenylyl, and a fused ring system optionally having a heteroatom selected from N, O and S;

W² is H;

Ar, R and n are defined as in formula (3); and

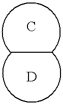

represents a fused ring system of 10 members, optionally containing 1 or 2 heteroatoms selected from N, O and S.

Compounds having formula (3), and (3A)-(3C) and methods for synthesizing such compounds are set forth in WO 02/22600, which is incorporated herein by reference.

Other CXCR4 antagonists have formula (4):

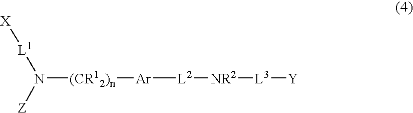

or the salts, prodrugs and stereochemical forms thereof, wherein:

X is a monocyclic (5-6 membered) or fused bicyclic (9-12 membered) unsubstituted or substituted ring system containing at least one heteroatom selected from N, O and S;

Z is H, or is an optionally substituted 5-6 membered monocyclic or 9-12 membered fused bicyclic ring system containing N, O or S;

Ar is an optionally substituted aromatic or heteroaromatic ring;

each of L¹, L² and L³ is independently a bond, CO, SO₂, or CH₂, wherein at least one of L² and L³ must comprise CO or SO₂; and wherein L¹ can also be alkylene (2-5C) wherein one or two C may optionally be replaced by N and which alkylene may itself optionally be substituted by a bridge alkylene (3-4C); L² and L³ also may be, independently, SO₂NH, CONH, SO₂NHCH₂ or CONHCH₂;

n is 0, 1 or 2;

each R¹ and R² is independently H or straight or branched chain or cyclic alkyl (1-6C) which may optionally be substituted, and wherein R² may be alkylene coupled to Y; and

Y comprises at least one aromatic or heteroaromatic or other heterocyclic substituted or unsubstituted ring coupled directly to L³.

In the above formula (4), X may be dihydroquinoline, tetrahydroquinoline, pyranopyridine, dihydropyranopyridine, thiapyranopyridine, dihydrothiapyranopyridine, dihydronaphthyridine, tetrahydronaphthyridine, imidazolyl, oxazolyl, thiazolyl, benzimidazolyl, benzothiazolyl, or benzoxazolyl.

In the above formula (4), L¹ may be alkylene (2-5C) wherein one C may optionally be replaced by N and which may optionally be substituted by a bridging alkylene (3-4C). For example, L¹ may be alkylene, CO or SO₂, and X is an optionally substituted imidazole, oxazole, thiazole, benzimidazole, benzothiazole, or benzoxazole. Alternatively, L¹ may be a bond, and X is substituted or unsubstituted dihydroquinoline, tetrahydroquinoline, pyranopyridine, dihydropyranopyridine, thiapyranopyridine, dihydrothiapyranopyridine, dihydronaphthyridine, or tetrahydronaphthyridine.

In the above formula (4), Z may be hydrogen.

In the above formula (4), Y may be an optionally substituted imidazole, benzimidazole, pyridine, pyridine, pyrimidine, or phenyl, wherein the ring nitrogen may optionally be oxidized. For example, Y may be substituted with halogen, nitrile, alkyl, —OR, —SR, —NR₂, —NRCOR, —OOCR, —COR, —CONR₂, —COOR, —NO₂, —NOH, —CF₃, where R is H or alkyl (1-6C).

In the above formula (4), each X or Z may optionally be substituted by halo, nitro, cyano, carboxy, C1-10 alkyl, C2-10 alkenyl, C3-10 cycloalkyl, hydroxy, thiol, amino, acyl, carboxylate, carbamate, carboxamide, sulfonamide, a carbonyl or sulfonyl binding to a hydrogen, or substituted with a C1-10-alkyl, C2-10 alkenyl, C3-7 cycloalkyl or a 5-6 membered monocyclic aromatic group; or X or Z may optionally be substituted by a 5-6 membered monocyclic aromatic group, naphthyl or a 5-6 membered heterocyclic ring;

Other CXCR4 antagonists have formula (4A):

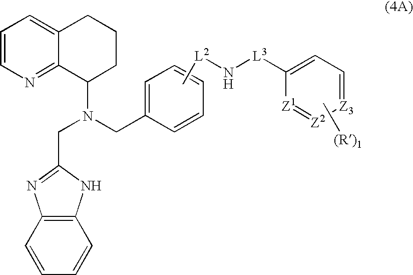

or formula (4B):

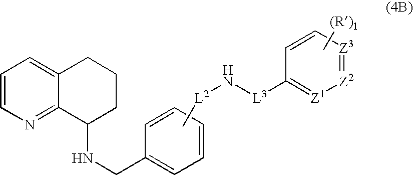

wherein 1 is 0-3, and R′ is OH, MeO, SH SMe, CN, CO₂Me, F, Cl, Br, NO₂, CH₃CO, NH₂, NHCH₃, N(CH₃)₂, CH₃CONH, CH₃SO₂NH, CONH₂, SO₂NH₂, CF₃, or Me;

each of Z¹, Z² and Z³ is independently CH, CR′ or N, wherein only two of said Z¹, Z² and Z³ can be N;

and L² and L³ are as defined in formula (4).

In the above formula (4A) or (4B), all of Z¹, Z² and Z³ may be CH or CR′. In one example, Z³ is N and L³ is CO. Furthermore, one of L² and L³ may be SO₂ and the other is a bond or CH₂. Alternatively, one of L² and L³ is CO and the other is a bond or CH₂.

In another embodiment, the compound for use in the methods of the present invention has formula (4C):

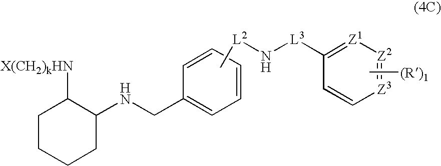

wherein 1 is 0-3, and R′ is OH, MeO, SH SMe, CN, CO₂Me, F, Cl, Br, NO₂, CH₃CO, NH₂, NHCH₃, N(CH₃)₂, CH₃CONH, CH₃SO₂NH, CONH₂, SO₂NH₂, CF₃, or Me;

k is 0-2;

each of Z¹, Z² and Z³ is independently CH, CR′ or N, wherein only two of said Z¹, Z² and Z³ can be N;

and X, L² and L³ are as defined in formula (4).

In the above formula (4C), all of Z¹, Z² and Z³ may be CH or CR′. In one example, Z³ is N and L³ is CO. Furthermore, one of L² and L³ may be SO₂ and the other is a bond or CH₂. Alternatively, one of L² and L³ may be CO and the other is a bond or CH₂.

Compounds having formula (4), and (4A)-(4C) and methods of synthesizing such compounds are set forth in WO 02/22599, which is incorporated herein by reference.

Other CXCR4 antagonists have formula (5):

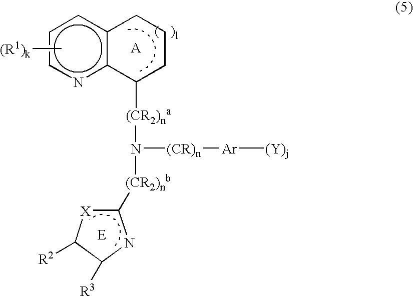

or the salts, prodrugs and stereoisomeric forms thereof;

Ring A optionally comprises a heteroatom selected from N, O and S;

the dotted lines represent optional unsaturation;

R¹, R² and R³ are independently H, halo, substituted or unsubstituted alkyl, hydroxyl, amino, thiol, or acyl; or R² and R³ may together form a benzo ring;

k is 0-4;

l is 0, 1, or 2;

X is unsubstituted or substituted C or N; or is O or S;

Ar is the residue of an aromatic or heteroaromatic moiety;

each n is independently 0-2;

each R is independently H or alkyl (1-6C);

j is 0-3; and

each Y is independently selected from the group consisting of halo, OR; SH; SO; SO₂;

optionally substituted phenyl;

(CR₂)ₘOR;

(CR₂)ₘCOR;

(CR₂)ₘCOOR;

(CR₂)ₘN═CH—NR₂;

(CR₂)ₘCONHNHR;

(CR₂)ₘCN;

(CR₂)ₘNR⁵₂;

(CR₂)ₘNR(CR₂)ₘNRR⁴;

(CR₂)ₘNR(CR₂)ₘNR(CR₂)ₘNR⁵₂;

(CR₂)ₘCO(CR₂)ₘNR⁵₂;

(CR₂)ₘCO(CR₂)ₘNR(CR₂)ₘNRR⁴;

(CR₂)ₘCO(CR₂)ₘNR(CR₂)ₘNR(CR₂)ₘNR⁵₂;

(CR₂)ₘNRCO(CR₂)ₘNRR⁴;

(CR₂)ₘNRCO(CR₂)ₘNR(CR₂)ₘNR⁵₂;

(CR₂)ₘNRCO(CR₂)ₘNR(CR₂)ₘNR(CR₂)ₘNR(CR₂)ₘNR⁵₂;

(CR₂)ₘNROH;

(CR₂)ₘCONROH;

(CR₂)ₘCR═NOH;

NHNHR;

CH═N-Z; and

guanidino or amidino, each of which may be linked to Y through a (CR₂)ₘ moiety;

wherein R is H or alkyl (1-6C), each m is independently 0-4, and each R⁴ and each R⁵ is independently H, alkyl (1-6C), alkenyl (2-6C), alkynyl (2-6C), or acyl (1-6C), each optionally substituted by one or more nonaromatic, nonheterocyclic substituent(s), wherein two R⁵ may be connected to form a cyclic amine optionally containing one or more additional heteroatoms selected from N, O and S;

a indicates the linker between Ring A and N;

b indicates the linker between ring E and the N; and

wherein Z is an aromatic or heteroaromatic moiety containing 5-12 ring members.

In the above formula (5), Ar may be a 5-6 membered monocyclic ring or a 9-12 membered fused ring system. For example, Ar may be benzene, naphthalene, dihydronaphthalene, tetrahydronaphthalene, pyridine, pyrimidine, quinoline, isoquinoline, imidazole, benzimidazole, azabenzimidazole, benzotriazole, furan, benzofuran, thiazole, benzothiazole, oxazole, benzoxazole, pyrrole, indole, imidazole, tetrahydroquinoline, tetrahydroisoquinoline, pyrazole, thiophene, isoxazole, isothiazole, triazole, tetrazole, oxadiazole, thiadiazole, imidazoline, and benzopyran. In particular examples, Ar is benzene, benzimidazole, benzothiazole, imidazole, oxazole, benztriazole, thiazole, pyridine, or pyrimidine. In one embodiment, at least one Y is —(CR₂)ₘNR⁵₂.

In the above formula (5), R² and R³ taken together may form a benzo substituent. In one embodiment, X is N and ring E comprises a pi bond coupled to one N. In one embodiment, ring E is coupled to the remainder of the molecule at position 2.

In the above formula (5), ring A may be saturated and l is 1. In one example, k is 0-1. In other examples, the ring system which includes A is tetrahydroquinoline or a substituted form thereof.

In the above formula (5), one of (CR₂)^{a}ₙ and (CR₂)^{b}ₙ may be CH₂ and the other is a bond. For example, (CR₂)^{a}ₙ may be a bond and (CR₂)^{b}ₙ is CH₂.

Compounds having formula (5) and methods for synthesizing such compounds are set forth in WO 02/34745, which is incorporated herein by reference.

Other CXCR4 antagonists have formula (6):

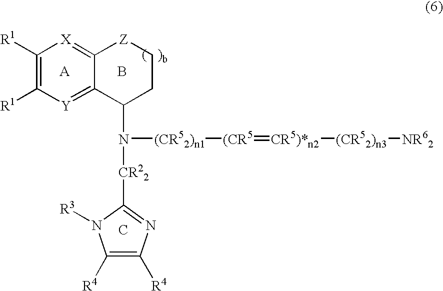

or the salts, prodrugs and stereoisomeric forms thereof,

wherein X and Y are independently N or CR¹;

Z is S, O, NR¹ or CR¹₂;

each R¹-R⁶ is independently H, halo, O(C═O)R, NR(C═O)R, OR, SR, NR₂, COOR, CONR₂, where R is H or optionally substituted alkyl, alkenyl, alkynyl or aryl; or

each R¹-R⁶ is alkyl (C₁₋₁₀), alkenyl (C₂₋₁₀), alkynyl (C₂₋₁₀), aryl (C₅₋₁₂), arylalkyl, arylalkenyl, or arylalkynyl, each optionally containing substituted and optionally containing O, S, or N; or an optionally substituted acyl, arylacyl, alkyl-alkenyl-, alkynyl- or arylsulfonyl wherein each alkyl, alkenyl, alkynyl or aryl moiety may contain O, O or N;

n1 is 0-4;

n2 is 0-1, wherein the * signifies C≡C may be substituted for CR⁵═CR⁵;

n3 is 0-4;

wherein n1+n2+n3 is greater than or equal to 2;

b is 0-2;

wherein the following combinations of R groups may be coupled to generate a ring, which ring may be saturated or unsaturated:

R²+R²

one R²+R³

R³+one R⁴,

R⁴+R⁴,

one R⁵+another R⁵,

one R⁵+one R⁶, and

R⁶+R⁶;

wherein the ring may not be aromatic when the participants in ring formation are two R⁵; and

wherein when n2 is 1, neither n1 nor n3 can be 0.

Other CXCR4 antagonists have formula (6A):

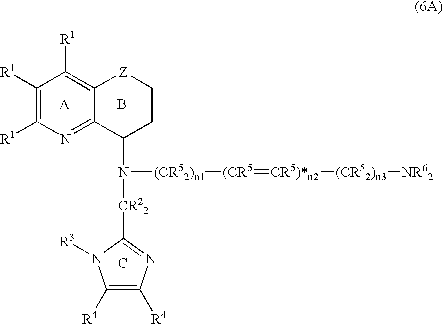

or the salts, prodrugs and stereoisomeric forms thereof,

wherein R¹-R⁶ and n1-n3 are as defined in formula (6).

Other antagonists have formula (6B) or formula (6C):

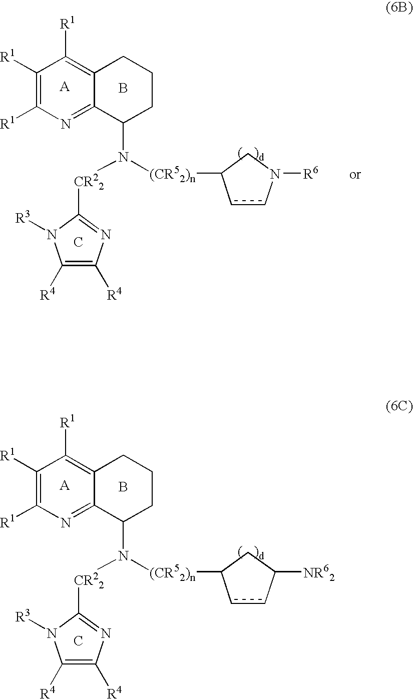

or the salts, prodrugs and stereoisomeric forms thereof,

wherein n is 0-1;

d is 0-3; the dotted line is an optional π bond; and

R¹-R⁶ are defined as in formula (6).

In yet another embodiment, the compounds for use in the methods of the present invention have formula (6D):

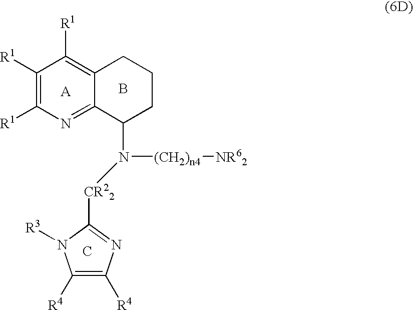

or the salts, prodrugs and stereoisomeric forms thereof,

wherein R¹-R⁶ are defined as in formula (6), and n4 is 2-6.

In the above formula (6) or (6A)-(6D), each R¹ may be H, halo, alkyl, alkoxy, or CF₃. In one embodiment, each R² is H or alkyl. In another embodiment, each R³ is H, alkyl, alkenyl, arylalkyl, or aryl.

In the above formula (6) or (6A)-(6D), each R⁴ may, be H, alkyl or aryl. Alternatively, two R⁴ may form an optionally substituted aromatic or heteroaromatic ring. For example, two R⁴ may form a phenyl or pyridyl ring, which may be substituted with halo, alkyl, halogenated alkyl, hydroxy, or alkoxy.

In the above formula (6) or (6A)-(6D), each R⁵ may be H, alkyl, or alkenyl, wherein said alkyl or alkenyl may optionally be substituted. In one embodiment, the alkyl or alkenyl substituents on a single carbon, or on nonadjacent or adjacent carbons, form a saturated or unsaturated ring. In one example, the substituents form a nonaromatic ring. In another embodiment, one R⁵ is an oxime, an alkylated oxime, alkylated hydroxylamine, hydroxylamine or halo.

In the above formula (6) or (6A)-(6D), each R⁶ may independently H, or an arylalkyl or arylsulfonyl, wherein the aryl moiety may comprise a heteroatom; or two R⁶ may comprise a guanidyl, carbonyl, or carbamino group. In one embodiment, two R⁶together, or one R⁵ and one R⁶ together may form a saturated, unsaturated or aromatic ring, wherein each ring may optionally contain N, S or O.

Compounds having formula (6) and methods for synthesizing such compounds are set forth in WO 03/055876, which is incorporated herein by reference.

The CXCR4 antagonist may have formula (7):

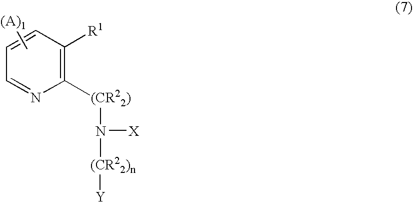

or the salts, prodrugs and stereoisomeric forms thereof,

wherein X is (CR³₂)ₒ—(CR³═CR³)ₚ—(CR³₂)_{q}—NR⁵₂; (CR³₂)ᵣ—R⁴; or an optionally substituted benzyl, or a monocyclic or bicyclic ring optionally containing N, O or S;

Y is an optionally substituted 5-12 membered heterocyclic ring containing a nitrogen atom, said heterocyclic ring may be monocyclic or fused, and is aromatic or partially aromatic;

A and R¹ are independently halo, CF₃, cyano, nitro, OR, SR, NR₂, COOR, CONR₂, NSO₂R, OSO₂R, or OSO₂NR, where each R is H, alkyl, alkenyl, alkynyl or aryl; or A and R¹ are independently an optionally substituted alkoxy (C₁₋₁₀), alkyl (C₁₋₁₀), alkenyl (C₂₋₁₀), alkynyl (C₂₋₁₀), aryl (5-12 members), arylalkyl, arylalkenyl, or arylalkynyl, each of which may optionally contain O, S, or N;

R² and R³ are independently H or an optionally substituted alkyl;

R⁴ is an optionally substituted heterocyclic ring or heteroaryl; or R⁴ comprises a urea, hydroxyurea, sulfamide, acetamide, guanidine, cyanamide, hydroxylamine, cyanamide, imidazolidine-2-one, or a nicotinamide moiety, each of which may be substituted with a heterocyclic ring;

R is H or alkyl;

l and n are independently 0-4;

p is 0-1;

o and q are independently 1-4; and

r is 1-6.

In the above formula (7), at least one of R¹ and R² may not be H, and may be connected to form an additional ring such as an aryl or heteroaryl. In one example, two As may not form an additional ring. In another example, X is (CR³₂)ᵣ—R⁴, r is at least two, and R⁴ is 2-pyridinyl, quinolinyl, imidazolyl or furan.

In the above formula (7), X may be (CR³₂)ₒ—(CR³═CR³)ₚ—(CR³₂)_{q}—NR⁵₂, wherein each R³ and R⁵ are independently H and p may be zero. In particular embodiments, o and q together are 2-6. Alternatively, X may be (CR³₂)ₚ—R⁴, wherein R⁴ is a heterocyclic ring or heteroaryl, each of which contains a nitrogen atom. For example, R⁴ may be azetidine, pyrrolidinyl, pyridinyl, thiophenyl, imidazolyl, or benzimidazolyl. Alternatively, X may be a monocyclic or bicyclic ring optionally containing N, O or S, such as cyclohexyl, piperidine, 8-aza-bicyclo[3.2.1]octane or 3-aza-bicyclo[3.2.1]octane. In yet another embodiment, X is an optionally substituted benzyl, particularly a disubstituted benzyl.

In the above formula (7), Y may be a 5-6 membered heterocyclic ring containing a nitrogen atom adjacent to the atom that is attached to the remainder of the molecule. The 5-6 membered heterocyclic ring may be fused to another ring. For example, Y may be pyridine, pyrimidine, pyrazine, indole, benzimidazole, benzothiazole, imidazole, isoquinoline, tetrahydroquinoline, pyridazine, thiazole, or benzoimidazole. In particular examples, Y is tetrahydroquinoline, particularly a 5,6,7,8 tetrahydroquinoline moiety, attached at position 8 to the remainder of the molecule.

In the above formula (7), each optionally substituted moiety may be substituted with a heteroatom, halo, CF₃, cyano, nitro, hydroxy, alkoxy, carbonyl, carboxy, amino, amido, imino, cyano, sulfonyl; C₁₋₆ alkyl or C₂₋₆ alkenyl each of which may contain N, O, or S; or substituted with aryl, heteroaryl, carbocyclic or heterocyclic ring, each of which may further be substituted with the same substituents.

Compounds having formula (7) and methods for synthesizing such compounds are set forth in WO 04/091518, which is incorporated herein by reference.

The CXCR4 antagonist may have formula (8)

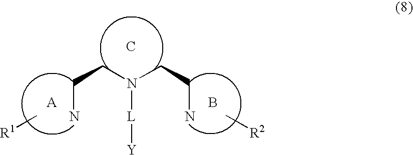

or the salts, prodrugs and stereoisomeric forms thereof,

wherein each of rings A and B is independently an optionally substituted 5-6 membered monocyclic heteroaryl;

ring C is an optionally substituted saturated or partially saturated 5-7 membered ring, and may contain a heteroatom in addition to nitrogen, wherein said heteroatom is N, O or S;

Y is H, a C₁₋₆ alkyl containing one or more heteroatoms, or a cyclic moiety, each of which is optionally substituted;

R¹ and R² are independently H, halo or an optionally substituted alkyl;

L is (CR³₂)ₗ or NR(CR³₂)ₗ wherein an alkyl bond may be replaced with an alkenyl or alkynyl bond;

l is 1-6; and

each R³ is H or alkyl.

In the above formula (8), at least one of R¹ and R² may not be H when C is piperidinyl or 1,2,3,6-tetrahydropyridinyl and rings A and B are pyridinyl. In other embodiments, R¹ and R² are not both naphthalenyl when ring C is piperidinyl and rings A and B are pyridinyl. In yet other embodiments, ring C is not 4-oxo-piperidine-3,5-dicarboxylic acid if L-Y is CH₃; and ring C is not 4-hydroxy-1,2,5,6-tetrahydro-pyridine-3-carboxylic acid ester if L-Y is benzyl.

In the above formula (8), R¹ and R² may be at positions adjacent the bonds to ring C. In one example, R¹ and R² are independently unsubstituted alkyl, such as methyl.

In the above formula (8), each of rings A and B may be pyridine, pyrimidine, pyrazine, pyridazine, 1,2,3-triazine, 1,2,4-triazine, 1,3,5-triazine, 1,2,4,5-tetrazine, pyrrole, imidazole, pyrazole, 1,2,3-triazole, 1,2,4-triazole, tetrazole, thiazole, oxazole, isothiazole, isoxazole, 1,2,3-thiadiazole, 1,3,4-thiadiazole, 1,2,3-oxadiazole, 1,3,4-oxadiazole, quinoline, isoquinoline, quinoxaline, quinazoline, phthalazine, cinnoline, 1,2,3-benzotriazine, 1,2,4-benzotriazine, indole, benzimidazole, 1H-indazole, benzoxazole, benzthiazole, benz[d]isoxazole, benz[d]isothiazole, or purine. In particular examples, each of rings A and B is pyridine, pyrimidine, imidazole, or benzimidazole, and each of rings A and B may be identical. Each of rings A and B may also contain a single substituent, which may be identical, at the position adjacent to the bond linking the rings to ring C.

In the above formula (8), ring C may be a saturated ring, or may contain a double bond. For example, ring C may be pyrrolidine, piperidine, hexahydro-1H-azepine, piperazine, morpholine, thiomorpholine, azepane, azocane, 2,3,4,7-tetrahydro-1H-azepine, 2,3,6,7-tetrahydro-1H-azepine, 3-pyrroline, 1,2,3,6-tetrahydropyridine, isoindoline, 1,2,3,4-tetrahydroisoquinoline, 2,3,4,5-tetrahydro-1H-benzo [d]azepine, 2,3,4,5-tetrahydro-1H-benzo[c]azepine, cyclobutane, cyclopentane, cyclohexane, cycloheptane, cyclooctane, cyclopentene, cyclohexene, cycloheptene, cyclooctene, tetrahydropyran, tetrahydrothiopyran, oxepane, thiepane, oxocane, or thiocane. In particular examples, ring C is pyrrolidine, piperidine, piperazine or hexahydro-1H-azapine. Ring C may be substituted with an optionally substituted alkyl, halo, cyano, oxime, OR or C═N—OR, wherein R is an optionally substituted alkyl.

In the above formula (8), Y may be selected from the group consisting of:

(CR₂)ₘNR₂,

(CR₂)ₘNR₂(CR₃),

(CR₂)ₘNR(CR₂)ₘNR₂,

(CR₂)ₘNR(CR₂)ₘNR(CR₂)ₘNR₂,

(CR₂)ₘOR,

(CR₂)ₘCO(CR₂)ₘOR,

(CR₂)ₘCO(CR₂)ₘNR₂,

(CR₂)ₘCO(CR₂)ₘNR(CR₂)ₘNR₂,

(CR₂)ₘNRCO(CR₂)ₘNR₂,

(CR₂)ₘNR(CR₂)ₘCO₂R,

(CR₂)ₘNR(CR₂)ₘCOR,

(CR₂)ₘNR(CR₂)ₘSO₂R,

(CR₂)ₘNRCO(CR₂)ₘNR(CR₂)ₘNR₂,

(CR₂)ₘNRCO(CR₂)ₘNR(CR₂)ₘNR(CR₂)ₘNR(CR₂)ₘNR₂,

(CR₂)ₘNR(CR₂)ₘOR,

(CR₂)ₘCR═NOH,

(CR₂)ₘCONR(CR₂)ₘOR,

(CR₂)ₘN[(CR₂)ₘCO₂R]₂,

(CR₂)ₘONRCONR₂,

—(CR₂)ₘ-Z

(CR₂)ₘNR—(CO)ₘZ,

(CR₂)ₘNR—(CR₂)ₘZ, and

(CR₂)ₘ—CR═N═Z;

wherein each R is H or an optionally substituted alkyl,

each m is independently 0-4; and

Z is an optionally substituted aromatic or heteroaromatic moiety containing 5-12 ring members.

In particular embodiments, Y is (CH₂)₁NR₂ and 1 is 1-10. Alternatively, Y may be a 5-12 membered aromatic, heteroaromatic, or a heterocyclic moiety, each of which may be a monocyclic or fused ring. For example, Y may be phenyl, imidazole, pyridine, thiophene, pyrrolidine, pyrazole, piperidine, azetidine, benzimidazole, benzo[d]isoxazole, or thiazole. Furthermore, Y may optionally be substituted with halo; cyano; nitro; alkoxy; halogenated alkyl; substituted carbonyl; a cyclic moiety such as a 5-12 membered aryl or heteroaryl containing N, O or S; or an alkyl, alkenyl, or a heteroalkyl moiety optionally containing one or more N, O, S, each of which is optionally substituted and optionally in the form of oxides. In particular examples, Y is substituted with pyridine, phenyl, piperidine or 2H-tetrazole.

In the above formula (8), each optionally substituted group may be substituted with inorganic moieties such as a heteroatom, halo, nitro, hydroxy, carboxy, amino, amido, cyano, or sulfonyl; or may be substituted with alkyl (C₁₋₁₀), alkenyl (C₂₋₁₀), alkynyl (C₂₋₁₀), aryl (5-12 members), arylalkyl, arylalkenyl, and arylalkynyl, each of which may optionally contain a heteroatom such as O, S, or N, and each of which may further be substituted with the same substituents. For example, each optionally substituted alkyl may be substituted with a heteroatom such as N, O, or S, or with a carbocyclic, heterocyclic, aryl or heteroaryl substituent.

Compounds having formula (8) and methods for synthesizing such compounds are set forth in WO 04/093817, and in U.S. patent application published as US 2005/0154201, each of which is incorporated herein by reference.

It is understood that the foregoing detailed description and accompanying examples are merely illustrative, and are not to be taken as limitations upon the scope of the invention. U.S. patents and publications referenced herein are incorporated by reference.

1. A method to enhance the effectiveness of a chemotherapeutic method in a subject afflicted with a hematopoietic or myeloid malignancy, which method comprises administering a chemotherapeutic method to said subject along with
administering to said subject an amount at least one CXCR4 antagonist
which is effective to enhance the effectiveness of said chemotherapeutic method.

2. The method of claim 1 wherein the CXCR4 antagonist is of the formula
Z-linker-Z′ (1)
or pharmaceutically acceptable salt or prodrug forms thereof
wherein Z is a cyclic polyamine containing 9-32 ring members of which 2-8 are nitrogen atoms, said nitrogen atoms separated from each other by at least 2 carbon atoms, and wherein said heterocycle may optionally contain additional heteroatoms besides nitrogen and/or may be fused to an additional ring system;
or Z is of the formula 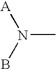
wherein A comprises a monocyclic or bicyclic fused ring system containing at least one N and B is H or an organic moiety of 1-20 atoms,
Z′ may be embodied in a form as defined by Z above, or alternatively may be of the formula
—N(R)—(CR₂)ₙ—X
wherein each R is independently H or straight, branched or cyclic alkyl (1-6C), n is 1 or 2, and X is an aromatic ring, including heteroaromatic rings, or is a mercaptan,
or Z′ is absent;
“linker” represents a bond, alkylene (1-6C) or may comprise aryl, fused aryl, oxygen atoms contained in an alkylene chain, or may contain keto groups or nitrogen or sulfur atoms.

3. The method of claim 2 wherein Z and Z′ are both cyclic polyamines.

4. The method of claim 3 wherein the compound of formula (1) is 1,1′-[1,4-phenylene-bis-(methylene)-bis-1,4,8,11-tetraazacyclotetradecane (AMD3100).

5. The method of claim 2 wherein Z is a cyclic polyamine and Z′ is N(R)—(CR₂)ₙ—X.

6. The method of claim 5 wherein each R is H, n is 2 and X is substituted or unsubstituted pyridyl.

7. The method of claim 6 wherein the compound of formula 1 is N-[1,4,8,11-tetraazacyclotetradecanyl-(1,4-phenylene-bis-(methylene)]-2-aminoethyl-2-pyridine (AMD3465).

8. The method of claim 2 wherein
Z is of the formula 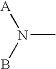
wherein A comprises a monocyclic or bicyclic fused ring system containing at least one N and B is H or an organic moiety of 1-20 atoms.

9. The method of claim 8 wherein A is 5,6,7,8-tetrahydroquinolin-8-yl and B is 1H-benzimidazol-2-yl methyl.

10. The method of claim 9 wherein Z′ is absent and the linker is an omega aminoalkyl substituent.

11. The method of claim 10 wherein the compound of formula 1 is N¹-(1H-benzimidazol-2-yl methyl)-N¹-(5,6,7,8-tetrahydroquinolin-8-yl)butane-1,4-diamine (AMD11070).

12. The method of claim 2 wherein the compound of formula (1) is administered to said subject in the dosage range of about 0.1 μg/kg-5 mg/kg of body weight.

13. A pharmaceutical composition comprising an effective amount of a CXCR4 antagonist in unit dosage form for enhancing the effectiveness of a chemotherapeutic method in a subject.
